(19) United States
(12) Patent Application Publication
(10) Pub. No.: US 2026/0182945 A1
(43) Pub. Date: Jul. 2, 2026

(54) AUTOMATED MODULATION OF RADIATION DOSAGE BASED ON AI DETECTION OF OBFUSCATING NOISE AND BEHAVIOUR

(71) Applicant: KONINKLIJKE PHILIPS N.V., EINDHOVEN (NL)

(72) Inventors: Brian Curtis LEE, ROCKVILLE, MD (US); Javad FOTOUHI, CAMBRIDGE, MA (US); Amin FEIZPOUR, CAMBRIDGE, MA (US); Ayushi SINHA, CLEVELAND HEIGHTS, OH (US); Leili SALEHI, WALTHAM, MA (US)

(21) Appl. No.: 19/132,050

(22) PCT Filed: Nov. 24, 2023
(86) PCT No.: PCT/EP2023/082974

§ 371 Date: May 22, 2025

Related U.S. Application Data
(60) Provisional application No. 63/430,690, filed on Dec. 7, 2022.

(51) Int. Cl. A61B 6/00

(52) U.S. Cl. CPC A61B 6/487; A61B 6/5264; A61B 6/542

(57) ABSTRACT

A system (30) for modulating radiation dosage during an x-ray guided procedure with an x-ray imaging system (50) comprises a detector (32) and a controller (74). The detector (32) is configured to detect at least one noise-causing event or user behavior event during the x-ray guided procedure with the x-ray imaging system (50), that renders an image quality for x-ray imaging above a threshold image quality unnecessary. The controller (74) is configured to automatically modulate x-ray system parameters that affect image quality and radiation dose based on the detection of the at least one of the noise-causing event or user behavior event.

### BACKGROUND

The present embodiments relate generally to x-ray image guided minimally invasive procedures and more particularly, to a system for automated modulation of radiation dosage based on AI detection of obfuscating noise and behavior and a method thereof.

During x-ray image guided minimally invasive procedures, the use of fluoroscopy is manually controlled by the operating physician. Generally, this control is binary—the x-ray is either on or off depending on the user's interaction with a single pedal/button on the system, and the interventionalist does not have easy access to fine-grained control of the x-ray system properties, nor would they have the bandwidth to manipulate these fine-grained controls during a procedure. Hence, the system is expected to produce a high-quality image any time the pedal/button is depressed. Quality may be defined in several ways, including spatial resolution, temporal resolution, noise reduction, etc. However, a high-quality image may not be necessary throughout an entire, interventional procedure.

As a result, the radiation dose delivered to both the patient and the interventionalist may be higher than necessary at times during the procedure. Interventionalists, in particular, may have high radiation doses delivered to their hands over the course of a career, and patients may be exposed to extended use of fluoroscopy, especially during x-ray-guided minimally invasive procedures; for instance, coronary angiography is reported to deliver a dose of approximately 5-15 milli-sieverts (mSv), the equivalent of approximately 2-5 years of background radiation. High radiation doses are associated with increased risk of excess cancer, and thus it would be desirable to reduce radiation exposure to both patients and interventional staff.

There is a trade-off between the radiation dose and image quality. In general, reducing radiation also reduces image quality in some way. For instance, the x-ray system frame rate is effectively proportional to dose—reducing the frame rate degrades the temporal image quality by updating the view less often, using fewer x-ray pulses/second thereby reducing the delivered radiation dose. In another example, increasing only the voltage (kV, or peak tube voltage kVp) of the x-ray source increases the penetration of the x-ray beam, thereby increasing the intensity of the signal measured by the x-ray detector and also the radiation dose delivered to the patient's tissue. However, in practice, when peak tube voltage kVp is increased, mAs (x-ray tube current*exposure time) is generally decreased to compensate for the increased number of photons reaching the x-ray detector, which results in an overall decrease in delivered radiation dose.

Additionally, when peak tube voltage kVp is increased, the amount of scatter is also increased due to an increased likelihood of photon interactions, resulting in reduced image clarity. In practice, acquiring a higher-energy radiograph results in a tradeoff of reduced radiation and increased penetration versus increased scatter. FIG. 1 provides an image view illustration of an example of energy-dose tradeoff. The left image 10 is acquired at a higher dose than the right image 12 (i.e., lower peak tube voltage kVp, but higher mAs (x-ray tube current*exposure time)) and exhibits less scatter, resulting in an image with improved clarity.

Other x-ray system properties may also have an effect on radiation dose, such as x-ray beam magnification. By combining x-ray beam magnification with collimation, it is possible to image the same field of view with a less focused beam, resulting in fewer photons reaching the x-ray detector. This effect may be interpreted as being similar to reducing the spatial resolution of the radiograph image as a tradeoff for reducing delivered radiation dose. In summary, there are multiple factors affecting image quality that can be modulated on the x-ray system to change the delivered radiation dose.

Currently, the state of the art for modulating radiation dose in x-ray systems is a set of tools known as “automatic dose rate control” (ADRC) or “automatic exposure control” (AEC). Existing ADRC systems control system parameters such as tube voltage, current, and exposure time in order to reduce radiation to the patient. Rudimentary ADRC systems terminate an exposure when a preset radiation level has been received at the detector, in order to maintain consistent intensity and signal-to-noise ratio across radiograph images and to ensure that an excessive radiation dose isn't delivered to the patient. More advanced ADRC systems in modern x-ray systems automatically measure the signal-to-noise ratio and the patient thickness and control x-ray tube parameters as the beam is moved. For instance, in the Philips Allura™ system, the ADRC estimates the approximate or equivalent water thickness of the patient given the gantry geometry. This estimate is updated based on pixel intensity values from previous runs and x-ray beam settings/geometry. Using the patient thickness as a starting point, the radiation dose is also controlled to maintain a consistent detector output and signal-to-noise ratio. Similar concepts also exist in computed tomography (CT) systems in the field; one example is a Smart mA feature implemented on a CT system which automatically modulates tube current over the course of a CT scan based on patient size and attenuation to maintain a specified noise level in the CT scan image.

Existing ADRC/AEC technologies disadvantageously focus on patient size and predetermined noise models to inform the modulation of dose control systems. However, it would be desirable to provide intelligent control of dose based on real-time feedback from the x-ray image itself, especially when it comes to detecting noise that results from physical events such as the movement of the patient, obscuration of the imaging field of view, or even behavior from the interventionalist or user that indicates that high image quality is not currently necessary.

Accordingly, an improved method and apparatus for overcoming the problems in the art is desired.

### SUMMARY

In accordance with one aspect, minimizing radiation delivered to the patient and the interventional staff during X-ray-guided procedures remains a critical task. Interventionalists (or interventional staff) can receive large doses of radiation over the course of a career and patients often receive large doses during a fluoroscopy-guided procedure, leading to increased risk of malignancy. A tradeoff exists between dose and image quality—reducing one may affect the other. X-ray can be used for extended periods of time during an intervention; however, its use is controlled manually in a binary fashion, and may not be well optimized. Images of high-quality are not necessarily required during all moments that the X-ray pedal (i.e., for activating X-ray exposure(s)) is being depressed. For instance, if the patient is moving, or an obstructing object obscures the field of view, a high resolution (spatial, temporal, etc.) view may not be needed. Similarly, if the physician is simply view-finding or looking at the gross anatomy rather than objects requiring precise vision, image quality may be less important than dose. According to one aspect, a method is disclosed herein to detect opportunities to automatically reduce the radiation output from the x-ray system in exchange for reducing image quality without disrupting the x-ray image guided procedure.

According to one embodiment, a system for modulating radiation dosage comprises a detector configured to detect at least one noise-causing event or user behavior event, during an x-ray image guided procedure with an x-ray imaging system, that renders an image quality for x-ray imaging above a threshold image quality unnecessary. The system further comprises a controller configured to automatically modulate x-ray system parameters that affect image quality and radiation dose based on the detection of the at least one of the noise-causing event or user behavior event. In one embodiment, the system includes wherein the detector comprises a noise detection model that is designed to detect the at least one noise-causing event or user behavior event, during the x-ray image guided procedure. In another embodiment, the controller is further configured to automatically modulate the x-ray system parameters according to a system settings model designed to control x-ray system settings based on an output from the detector.

According to one embodiment, the system for modulating radiation dosage is trained by: receiving X-ray image data containing noise from a noise-causing event, inputting received X-ray image data into the noise detection model, generating a predicted level of noise in the inputted image using the noise detection model, inputting the predicted level of noise into the system settings model, generating a predicted X-ray system parameters using the system settings model, adjusting parameters of the noise detection model, or the system settings model, or both, based on a comparison between: (i) the predicted level of noise with an expected level of noise, or (ii) a simulated image generated from the predicted X-ray system parameters with an expected X-ray image, or (iii) a combination thereof, and repeating the inputting, the generating, and the adjusting, until a stopping criterion is met.

According to another embodiment, the noise detection model includes a noise classifier H(x) configured for detecting noise levels from different noise sources, wherein noise of the noise levels refers to any source of obfuscation in a current x-ray image of the x-ray image guided procedure. The noise classifier H(x) is trained via producing a training data set in which known motion, obscuration, user behaviors, and other augmentations were introduced while using the x-ray imaging system.

In yet another embodiment, the controller is further configured to automatically modulate the x-ray system parameters according to a system settings model, wherein the system settings model is designed to control x-ray system settings according to a set of predicted optimal x-ray system settings based on an output from the noise classifier H(x) of the noise detection model, wherein the image quality is reduced to a visually perceivable threshold level that is indistinguishable or nearly indistinguishable from the original image in the presence of the detected noise. In one embodiment, the system settings model comprises at least one selected from the group consisting of (i) a direct noise level to system setting mapping function that directly maps a set of detected noise levels to the set of predicted optimal x-ray system settings, (ii) a semi-supervised image based mapping of noise level to system settings that is trained without directly labelling ideal x-ray system settings for each input training image or behavior, and (iii) a combination thereof, to provide the set of predicted optimal x-ray system settings.

In another embodiment, the detector is further configured to predict a user behavior, indicative of a current procedural phase or event of the x-ray-guided procedure being performed via the x-ray imaging system, that does not require an x-ray image having a quality greater than a threshold image quality, based upon information inputs from (i) the x-ray imaging system and (ii) an operating room in which the x-ray imaging system is located, the information inputs including at least one of (a) the current x-ray image, (b) x-ray imaging system user interaction information, and (c) operating room sensor/camera data.

In yet another embodiment, the noise detection model takes as input a series of x-ray images in a live fluoroscopy run as individual images of the series are acquired, and considers the series of x-ray images in parallel with each newly acquired x-ray image in order to better predict changes in behavior, motion, or other temporal variables. In a further embodiment, an output of the noise detection model is additionally dependent on a duration of time since any of the at least one noise-causing or user behavior event occurred, wherein modulations of the x-ray system parameters based on outputs of the noise detection model are implemented in real time during the detected at least one noise-causing or user behavior event and once a respective event is complete or no longer occurring and new x-ray images are being acquired sometime later, an influence of the respective event on the x-ray system settings for new x-ray image acquisition is lower, having decayed with time, wherein the controller reverts modulated x-ray system parameters back to parameters which existed prior to a respective modulation.

According to one embodiment, the system includes at least one of (a) wherein the at least one noise-causing or user behavior event comprises (i) an obfuscation or motion noise-causing event or (ii) a user behavior event requiring an image quality less than or equal to the threshold image quality, associated with respect to a current x-ray image of the x-ray guided procedure, or (b) wherein the x-ray system parameters comprise at least one selected from the group consisting of x-ray tube voltage/peak tube voltage (kV/kVp), x-ray tube current (mA), exposure time, frame rate, magnification, and any combination thereof. In addition, the obfuscation noise-causing event can include at least an object obscuring a field of view of an x-ray tube of the x-ray imaging system, wherein the motion noise-causing event includes at least a patient or an x-ray tube or x-ray detector component movement, and wherein the user behavior event includes at least a view-finding event.

According to another embodiment, an x-ray imaging system comprises a detector configured to detect at least one noise-causing event or user behavior event, during an x-ray guided procedure, that renders an image quality for x-ray imaging above a threshold image quality unnecessary. The x-ray imaging system further comprises a controller configured to automatically modulate x-ray system parameters that affect image quality and radiation dose based on the detection of the at least one of the noise-causing event or user behavior event. In one embodiment, the detector comprises a noise detection model that is designed to detect the at least one noise-causing event or user behavior event, during the x-ray guided procedure. In addition, the controller is further configured to automatically modulate the x-ray system parameters according to a system settings model designed to control x-ray system settings based on an output from the detector.

Furthermore, the x-ray imaging system is trained by: receiving X-ray image data containing noise from a noise-causing event, inputting received X-ray image data into the noise detection model, generating a predicted level of noise in the inputted image using the noise detection model, inputting the predicted level of noise into the system settings model, generating a predicted X-ray system parameters using the system settings model, adjusting parameters of the noise detection model, or the system settings model, or both, based on a comparison between: (i) the predicted level of noise with an expected level of noise, or (ii) a simulated image generated from the predicted X-ray system parameters with an expected X-ray image, or (iii) a combination thereof, and repeating the inputting, the generating, and the adjusting, until a stopping criterion is met.

In accordance with a further embodiment, a method for modulating radiation dosage comprises: detecting at least one noise-causing event or user behavior event, during an x-ray guided procedure with an x-ray imaging system, that renders an image quality for x-ray imaging above a threshold image quality unnecessary; and automatically modulating x-ray system parameters that affect image quality and radiation dose based on the detection of the at least one of the noise-causing event or user behavior event. In one embodiment, the method further includes at least one of (a) wherein the detecting comprises detecting via a noise detection model that is designed to detect the at least one noise-causing event or user behavior event, during the x-ray guided procedure, or (b) wherein automatically modulating the x-ray system parameters comprises modulating according to a system settings model trained to control x-ray system settings based on an output from the detecting step.

According to yet another embodiment, a non-transitory computer-readable medium is encoded with computer program code that comprises a set of instructions, executable by a computer for enabling the computer to carry out the method to modulate radiation dosage based upon detection of at least one noise-causing event or user behavior event during performance of an x-ray-guided procedure with an x-ray imaging system.

Advantages and benefits will become apparent to those of ordinary skill in the art upon reading and understanding the following detailed description.

### BRIEF DESCRIPTION OF THE DRAWINGS

The embodiments of the present disclosure may take form in various components and arrangements of components, and in various steps and arrangements of steps. Accordingly, the drawings are for purposes of illustrating the various embodiments and are not to be construed as limiting the embodiments. In the drawing figures, like reference numerals refer to like elements. In addition, it is to be noted that the figures may not be drawn to scale.

FIG. 1 is an image view illustration of an example of energy-dose tradeoff;

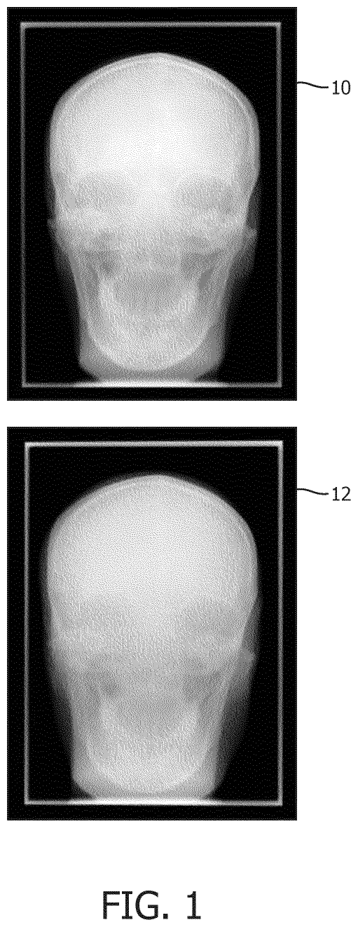

FIG. 2 is an image view illustration of an example of motion blur affecting image clarity, according to an embodiment of the present disclosure;

FIG. 3 is an image view illustration of examples of an effect of changing x-ray tube parameters to reduce radiation dose in the presence of motion blur, according to an embodiment of the present disclosure;

FIG. 4 is a block diagram view of a noise detection model that includes a noise classifier H(x) according to an embodiment of the present disclosure;

FIG. 5 is a block diagram view of a model to generate a new image given a change in x-ray imaging system settings according to an embodiment of the present disclosure;

FIG. 6 is a block diagram view of a generative adversarial semi-supervised training scheme for unlabeled data according to an embodiment of the present disclosure;

FIG. 7 is a block diagram view of an x-ray imaging system configured for modulating radiation dosage according to an embodiment of the present disclosure; and

FIG. 8 is a flow diagram view of a method for modulating radiation dosage according to an embodiment of the present disclosure.

### DETAILED DESCRIPTION

The embodiments of the present disclosure and the various features and advantageous details thereof are explained more fully with reference to the non-limiting examples that are described and/or illustrated in the drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the present disclosure. The examples used herein are intended merely to facilitate an understanding of ways in which the embodiments of the present may be practiced and to further enable those of skill in the art to practice the same. Accordingly, the examples herein should not be construed as limiting the scope of the embodiments of the present disclosure, which is defined solely by the appended claims and applicable law.

It is understood that the embodiments of the present disclosure are not limited to the particular methodology, protocols, devices, apparatus, materials, applications, etc., described herein, as these may vary. It is also to be understood that the terminology used herein is used for the purpose of describing particular embodiments only, and is not intended to be limiting in scope of the embodiments as claimed. It must be noted that as used herein and in the appended claims, the singular forms “a,” “an,” and “the” include plural reference unless the context clearly dictates otherwise.

Unless defined otherwise, all technical and scientific terms used herein have the same meanings as commonly understood by one of ordinary skill in the art to which the embodiments of the present disclosure belong. Preferred methods, devices, and materials are described, although any methods and materials similar or equivalent to those described herein can be used in the practice or testing of the embodiments.

According to one embodiment, a system is provided for detecting moments (e.g., detecting noise that results from physical events such as the movement of the patient, obscuration of the imaging field of view, or even behavior from the user that indicates that high image quality is not currently necessary) during an interventional x-ray guided procedure when high image quality is not required, such as when noise or other action obscures the x-ray detector's field of view. For example, if a sudden or fast motion occurs (i.e., if the patient moves or the c-arm moves), the human eye is not able to resolve small details in the radiograph or x-ray image regardless of image quality. In a non-medical example, when a dog jumps, one is not able to see the individual hairs on the dog's body due to the motion, hence a gigapixel image is not necessary. An analogous situation exists in medical imaging, where given a temporally or spatially chaotic scene, the user's eye would not be able to resolve small details of a corresponding medical image on the medical imaging system screen anyway.

In an x-ray imaging system according to an embodiment of the present disclosure, a neural network is trained to detect obfuscation or motion in the radiograph or x-ray image and determine whether or not the current image quality of the respective radiograph or x-ray image is appropriate based upon detection of one or more obfuscation/noise-causing events that may happen during the x-ray-guided fluoroscopy interventional procedure. A complex mapping between the x-ray system parameters, the resulting image quality, and the currently detected obfuscation level of the last x-ray image may be used to identify optimal x-ray system settings in the event that the radiation dose can be reduced without significantly affecting the ability to see objects in an updated x-ray image under new x-ray system settings.

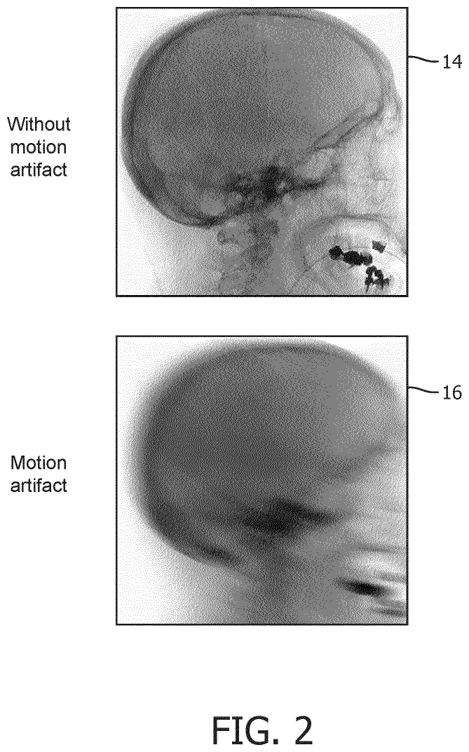

Turning now to FIG. 2, there is shown an image view illustration of an example of motion blur affecting image clarity, according to an embodiment of the present disclosure. The examples in FIG. 2 illustrate an image 14 without a motion artifact and an image 16 with a motion artifact. FIG. 2 further illustrates what the human eye would see in the presence of sudden or fast motion. In actuality, the image viewing screen of the x-ray imaging system may still be showing a high resolution and high-quality image, but the human eye may not be able to benefit fully from that image clarity.

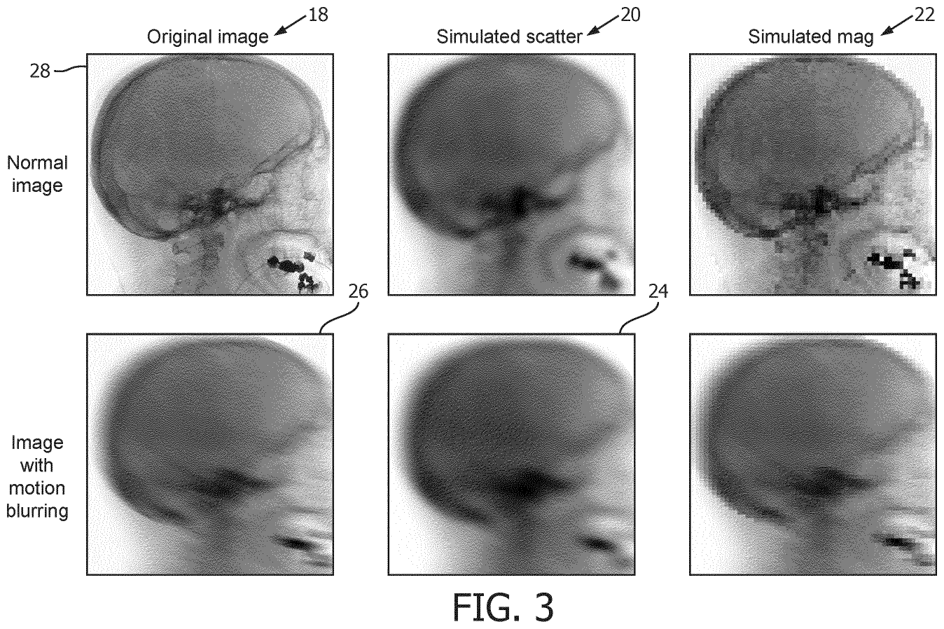

With reference now to FIG. 3, there is shown an image view illustration of several examples of an effect of changing x-ray tube parameters to reduce radiation dose in the presence of motion blur, according to an embodiment of the present disclosure. Comparing the left and center columns 18 and 20, respectively, the effect of simulating the same amount of scatter (center column 20, simulated blurring due to scattering) in the normal image and the motion blurred image illustrates the difference in loss of image clarity in the presence of noise. The difference between the motion blurred image with simulated scatter 24 and without simulated scatter 26 is barely perceptible, whereas the difference in the normal image 28 is clearly noticeable. This difference between the normal image 28 and motion blurred image 26 indicates that there may be some excess in radiation dose/image quality that can be taken advantage of in the presence of obfuscating noise. In the third column 22 of FIG. 3, the images represent a “Simulated Mag” image which refers to simulated magnification of the images of the first column 18. The effect in the images of column 22 is simply a reduction of image resolution. This occurs when digital zoom is used, although the field of view (FOV) would not stay the same as shown in the image. The images of column 22 are shown to provide a comparison between the same images of the first column 18. Alternatively, the third column may be referred to as a “reduction in resolution” instead of “simulated mag” to be more explicit.

One main problem addressed by the embodiments of the present disclosure is the limited ability of a physician to modulate the x-ray system parameters that affect radiation dosage in real time in response to obfuscation and motion noise-causing events that happen during the x-ray fluoroscopy procedure. Because of the frequency of obfuscation/noise-causing events, it is not realistic for a user to control voltage, current, exposure time, magnification, frame rate, and other various factors that affect both image quality and radiation dose. An artificial intelligence AI-enabled automatic method to modulate the image quality proportionally to the currently detected level of obfuscation in the image would advantageously reduce the radiation dosage without sacrificing image quality beyond the clarity possible given present conditions.

According to a first aspect, the method and system include a model or algorithm that is trained to detect obfuscation in the x-ray image or a user behavior (e.g., viewfinding) that renders high image quality unnecessary. According to a second aspect, the system includes a controller that acts in response to a detection by the model or algorithm by automatically modulating the x-ray system parameters in order to reduce image quality to the highest level reasonably able to be resolved by the human eye given the detection, which reduces the radiation dosage as a side effect. The x-ray system parameters include one or more of x-ray tube voltage (kV), peak x-ray tube voltage (kVp), x-ray tube current mA, exposure time, frame rate, magnification, etc.

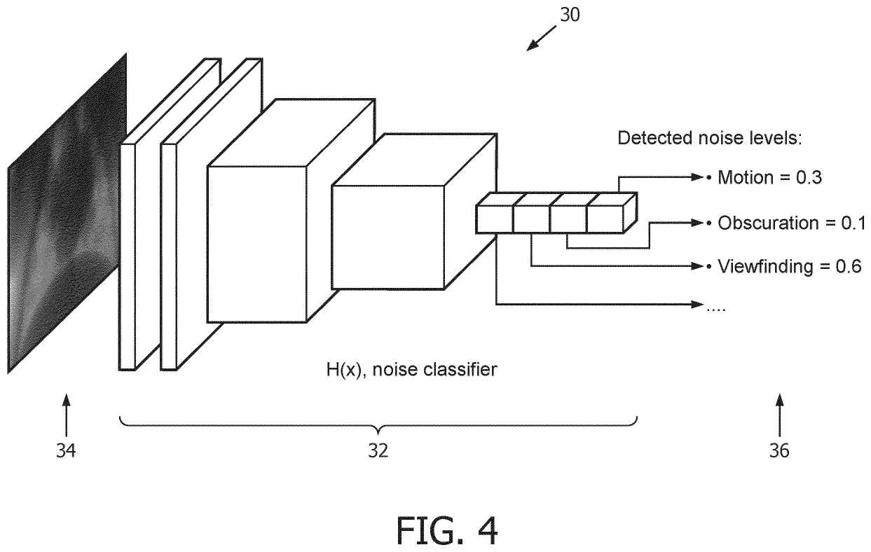

With reference now to FIG. 4, a block diagram view of a noise detection model 30 that includes a noise classifier H(x) 32 according to an embodiment of the present disclosure is shown. In one embodiment, in order to detect obfuscating noise or behavior, the noise detection model 30 comprises a neural network trained to receive inputs 34 including the x-ray images, system parameters, user inputs, and information from the operating room within which the x-ray imaging system is placed. Note that here, “noise” includes any source of obfuscation in the x-ray image as well as signal or measurement noise.

In one example, from a stream of acquired x-ray images, a neural network may extract image-based features that correlate with relevant sources of image obfuscation, such as detection of obfuscation by motion blurring. For instance, the neural network may be trained to detect a lack of sharpness in the edges of objects in a given x-ray image, potentially indicating motion blurring, or to digest a series of x-ray images in a recurrent neural network or transformer-like architecture to detect significant changes from frame to frame that would indicate fast motion. Computer vision methods such as optical flow may also be substituted to detect motion.

In addition or alternatively, live x-ray system settings/parameters may also be digested to detect obfuscation of the image. For instance, the combination of blurring/motion detected in the image, as described previously, such as with a detected fast movement of a patient table or c-arm from the system parameters or a longer exposure time from the system settings may indicate a high confidence of blurring caused specifically by motion. The speed of motion may also be inferred from the system parameters such as patient table movement or c-arm angulation, and therefore the amount of noise estimated due to motion may be increased when the speed of motion is increased. The output of the model or neural network may be a score (for instance, a scalar score between 0 and 1) that indicates the magnitude of the motion corresponding to an individual motion source, such as c-arm rotation, or the joint sum of all motion sources.

In another example, a neural network could be trained to detect obfuscation by the presence of an unexpected object in frame, such as a table, intravenous line (IV) line, ECG lead, or physician's hand in the x-ray beam path. An object detection neural network may be trained to identify unexpected objects in the received x-ray image and produce an estimate of how much the anatomy of interest has been obscured. Similarly, information extracted from the operating room may be used to detect physical objects blocking the x-ray beam. For example, an algorithm could use images from a camera in the operating room to detect the x-ray source and detector locations and angulation, as well as estimate whether or not any objects are lying in the path of the x-ray beam. In another example, the c-arm configuration may be extracted from the system settings and the known geometry of the room and compared to objects detected from a spatially calibrated camera system to detect intersections of the x-ray beam with unexpected objects.

In yet another example, a neural network may be configured to digest a combination of system settings/parameters, user inputs to the system, and information extracted from the operating room to detect user behavior indicating that high image fidelity is not required. For example, one instance when user behavior may indicate that high image quality is not required is when the user is trying to adjust the x-ray imaging system to find the best viewing angle/position for a particular step of a surgical procedure. Oftentimes during the viewfinding activity, high resolution image detail is not required and only a macroscopic view of the imaging target is needed to select the desired view. A neural network may be trained to detect specific interactions with the x-ray imaging system that indicate a high likelihood of viewfinding behavior. For instance, rapid panning of the c-arm/patient table, back-and-forth changes in c-arm angulation, or rapid changes in magnification/zoom may indicate that the user is engaged in viewfinding behavior. Similarly, information extracted from the operating room may augment the input to the neural network. For instance, tracking the eye-gaze of the user may indicate whether or not the user is focused on a specific part of the anatomy, in which case high image quality may be required, or if they are glancing quickly at multiple different parts of the image while changing the system parameters, in which case they may be engaged in viewfinding.

In still another example, a neural network may be configured to digest user interactions with the x-ray imaging system and information extracted from the operating room to detect user inattention. For instance, the user may be pressing the x-ray pedal but interacting with menu options on the system's user interface or their eye-gaze may be away from the screen entirely, indicating that the x-ray image being generated is not of high importance. The neural network may be trained to interpret a combination of inattentive eye-gaze and active user interaction with non-imaging aspects of the system (e.g., like scrolling through system menus) as an indication of a high likelihood of inattentiveness to the display screen.

Other than motion or physical obscuration or user behaviors, other noise sources that may be detected from a combination of the above inputs may include poor detector signal-to-noise ratio, overexposure in regions of an x-ray image, high degrees of scattering noise in the x-ray image, excessive blurring from non-motion sources (e.g., like focal spot blurring), excessive use of digital zoom, imaging target not centered in the field of view, and so on. Data inputs from different sources like the image stream, x-ray imaging system settings, etcetera, may have interactions that provide more robust noise detection.

As will be discussed further herein, the noise detection model or neural network 30 outputs a new set of system parameters that would appropriately change image quality and radiation dosage. The neural network may be trained in multiple parts. The illustration of the noise classifier H(x) in FIG. 4 includes several blocks which represent different downstream layers in a convolutional encoder, further which are part of the neural network. The blocks can vary in number/size depending on the network architecture of the given neural network. Neural networks are known in the art, and thus not described in further detail herein.

First, the classification network 32 detects noise levels from different noise sources, as shown in FIG. 4. The neural network may predict a probability of the presence of noise for each of a set of known noise types. For example, these noise types may include physical obscuration of the imaging target, motion, user inattentiveness, user viewfinding behavior, image overexposure, blurring from other sources, and so on. This noise classification network, H(x) 32, may be trained by producing a training data set in which known motion, obscuration, user behaviors, and other augmentations are introduced while using an x-ray imaging system. In one example, to produce a training dataset, x-ray images may be recorded while the c-arm is rotated at different speeds in order to indicate different levels of simple motion noise, where no c-arm rotation indicates 0% level of motion noise and maximum c-arm rotation speed indicates 100% level of motion noise. Of course, the network may be jointly trained on a number of different sources of motion, including patient motion, patient table motion, etcetera, in which case a similar score may be produced as a label indicating the magnitude of motion based on the joint combination of all inputs. At inference time, the neural network would receive a series of x-ray images and estimate the probability of motion noise in the new input based on the trained model. While the training data set would include a variety of inputs such as the x-ray images, system settings, user interactions, and information from the room, in the example of noise caused by excessive motion, a neural network trained to detect this type of noise would uncover a relationship between fast movements in subsequent x-ray images and system parameters indicating movement of the c-arm in order to detect motion.

In another example, to detect physical obscuration, a training dataset may be produced by recording x-ray images and operating room camera feed images of objects blocking the source-to-detector x-ray beam path. In the case of scoring physical obscuration of the image, a similar label may be produced by either manual scoring of the degradation of the image in the target region or quantifying the physical area of the target region being blocked by the offending object (for instance 0 to 100% where 100% indicates that the region of interest is completely blocked by an unexpected object). In FIG. 4, one example of detected noise level output 36 by the noise classifier H(x) may include motion (e.g., Motion=0.3), obscuration (e.g., Obscuration=0.1), viewfinding (e.g., Viewfinding=0.6), etc.

With respect to a translation of noise detection to system settings, the embodiments of the present disclosure provide several methods to train an algorithm or model to control the system settings of the x-ray imaging system based on the output from the noise classification network or noise classifier H(x) 32. The methods can include one or more of a first method of direct noise level to system setting mapping and a second method of semi-supervised image-based mapping of noise level to system settings. In another embodiment, a fully supervised training dataset can be produced to train the noise classifier.

For the method of direct noise level to system setting mapping according to one embodiment, a function can be provided which directly maps a set of detected noise levels to a set of optimal system settings. For instance, a set of probabilities/levels corresponding to a known set of noise types is produced by the noise classifier H(x) described above. The set of noise levels is fed as an input into a separate model whose role is to translate the noise levels into a change in x-ray imaging system settings that produces an image with the minimum possible radiation while imperceivably reducing the image quality given the presence of the detected noise. As an example, this separate model may be a simple predetermined mapping of one noise type to one system setting. In particular, the model may be designed such that detecting motion blur would be directly mapped to reducing peak tube voltage (kVp).

In another more complex example, a multivariate function maps multiple noise levels to multiple x-ray imaging system outputs or settings. Fitting this multivariate function would require a set of training data in which the optimal system settings corresponding to a particular noise level were annotated. The use of the term “annotated” here refers to some manual labeling of a training data set by an expert. In other words, for combining multiple noise factors, an expert may be shown an example of a noise-corrupted image, and may manually change x-ray system parameters in a direction that reduces radiation until the image begins to perceivably degrade. The maximum amount of change of parameters before the image starts to noticeably degrade would be the ground truth label or annotation. Depending on how the problem is formulated it may be valid for this annotation to be either a set of x-ray imaging system settings or an amount by which to change the x-ray system settings.

In yet another example, a polynomial function may be fit to the training data using the least squares method. In order to produce a training dataset to fit such a model in a supervised manner, a set of “output” imaging system settings would first be identified as having an impact on radiation dosage and image quality. Then, in one example, a dataset may be produced in which a known noise level is applied to an image (i.e., by manually introducing a known amount of motion noise or known amount of physical obscuration, and so on) and the set of “output” imaging system settings are modified to reduce radiation until the image quality begins to perceivably degrade in the presence of noise. The system settings at which the radiation dose is lowest before the image begins to perceivably degrade would then act as the dataset output while the noise level acts as the dataset input. These input and output pairs may be used to fit a simple model like a polynomial function or a more complex model like a neural network.

For instance, in a simplest embodiment with patient table motion as the only noise source and kVp/mAs as the only system settings to be modulated, a dataset may be produced where the patient table is translated in varying directions and speeds. For a particular resulting image series, the kVp and mAs settings may be modulated in order to reduce radiation dosage at the cost of increasing scatter (and therefore blurring) to the image. Due to the motion inherent in the dataset, the increased blurring due to system settings change may be imperceptible up to a certain level. This level may be determined by experimentation and used as a ground truth label to train the model to produce optimal system settings (or a change in system settings).

Similarly, to produce a dataset for training a model to translate noise levels related to user behavior into “output” system settings, a user may be directed to perform tasks like viewfinding while operating the x-ray imaging system. The viewfinding operation could be performed multiple times under different “output” system settings until the user identifies the system settings at which the image quality is not meaningfully degraded but radiation dosage is lowest. A similar data collection may be performed for the behavior of screen inattentiveness. Viewfinding and inattentiveness may be predicted by the noise classification network either as binary labels or as non-binary levels. As an example, to produce a dataset of non-binary user behaviors, the user may be asked or influenced to pay differing levels of attention to the screen.

For the method of semi-supervised image-based mapping of noise level to system settings according to another embodiment, training the algorithm or model in a semi-supervised manner is accomplished without directly labelling the ideal system settings for each input training image or behavior. For example, a second algorithm may be trained to predict the optimal system settings that reduce radiation dosage while not significantly degrading the x-ray image (to be acquired) beyond the degradation caused by the detected noise. In order to train this algorithm, an intermediate simulation model is employed that describes how a change in system settings would affect the x-ray image to be acquired, herein referred to as a “generator”.

This generator model takes as input the current system settings, the current x-ray image, and a set of new system settings and produces as output a new image that approximates the expected image given the new system settings. Note that the generator model is already trained or fitted and so its parameters are not modified during this process. This generator model may take the form of a physics-based model or a pre-trained neural network. For example, the generator model may take as input the current x-ray image and settings as well as an arbitrary new set of system settings where the kVp is increased and mAs is decreased (resulting in lower dose but higher scatter), and the generator model would output a new version of the x-ray image where the scattering is artificially increased as a result of modeling the effect of changing the x-ray system settings. The resulting image simulated by the generator is then compared to the original input image (or image series) by a discriminator model, whose role is to score the perceptibility of the change in image quality. When the currently selected change in system settings produces a dramatically and perceivably different image, the discriminator will score the output poorly (indicating that the user would be able to perceive the loss of image quality), and vice versa. The training is iterated until the system settings model is trained to produce the ideal set of system settings that rewards reduction in radiation but penalizes perceivable loss in image quality. The purpose of this intermediate model is to enable training a model to identify optimal change in system settings without a large manually labeled dataset of pairs of noise levels and corresponding ideal system settings.

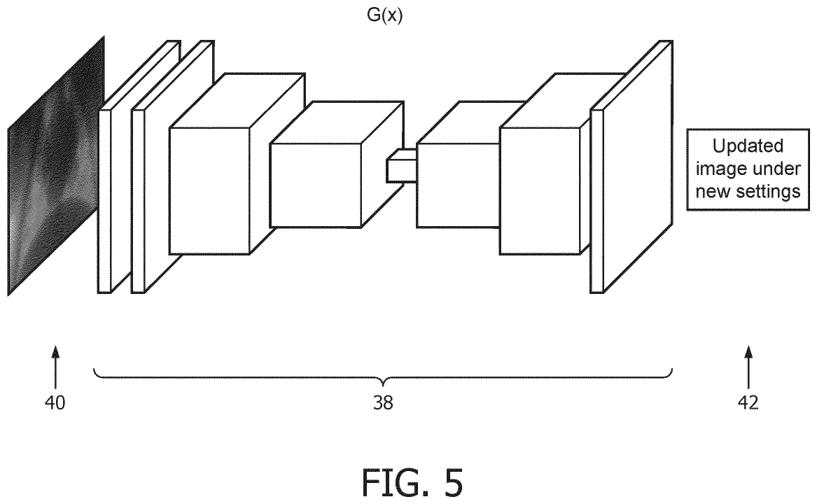

With reference now to FIG. 5, there is shown a block diagram view of a model 38 to generate a new image 42 given a change in system settings according to an embodiment of the present disclosure. In essence, the model G(x) 38 facilitates quantification of the resulting loss/increase in image quality based on the a given change in system parameters by simulating the resultant image, in order to assist with selecting the magnitude of each parameter change. The model 38 receives an input image 40 and outputs an updated or expected image 42 under new system settings. The illustration of the model G(x) in FIG. 5 includes eight blocks which represent different downstream layers in an encoder/decoder convolutional neural network. The blocks may vary in number/size depending on the network architecture of the given neural network. Neural networks are known in the art, and thus not described in further detail herein.

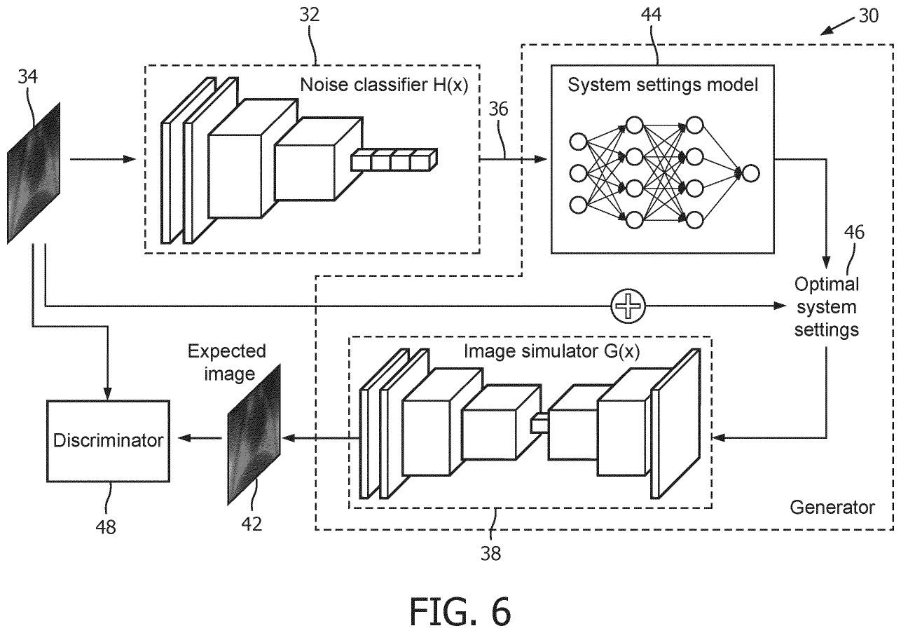

With reference now to FIG. 6, a block diagram view is shown of a generative adversarial semi-supervised training scheme for unlabeled data according to an embodiment of the present disclosure. Given a model G(x) 38 with fixed parameters after training, an additional module which contains a model 44 (i.e., Systems Settings Model) for mapping the detected noise levels from H(x) 32 to system settings is trained in an adversarial fashion without labeled training data containing the annotated optimal system settings. The system settings model parameters are optimized by feeding or inputting the predicted optimal system settings 46 into the image simulator G(x) 38 and the expected image output 42 is compared with the original image 34 by a discriminator D 48.

In an ideal scenario, the system settings 46 may be changed to reduce the radiation dose in a way that makes the resulting image 42 indistinguishable from the original image 34. Adversarial training is an example of training that would be ideal for this optimization. Here, the loss function to be optimized would be a combination of the discriminator's ability to distinguish the two images, the resulting decrease (or increase) in radiation dose, and the resulting change in noise level (which may be calculated using noise classifier H(x) 32). This workflow is illustrated in FIG. 6.

The loss functions to be minimized are:

$${loss}_{generator} = {E}_{generated} [ log ( 1 - D ( G ( x ) ) ) ] + {δ}_{noise} + {δ}_{dose} ,$$

$${loss}_{discriminator} = - {E}_{I} [ log D ( I ) ] - {E}_{gener ated} [ log 1 - D ( G ( x ) ) ] ,$$

where I is a set of original images, x is a set of system settings predicted from the output of noise classifier H(x), G(x) are a set of modified images (i.e., expected images), D is the discriminator output, and δₙₒᵢₛₑ and δ_{dose} are the change in noise level and radiation dose.

With respect to semi-supervised training, in one embodiment, automatically modulating the x-ray system parameters further comprises predicting optimal x-ray system settings that reduce radiation dosage while not significantly degrading a subsequent acquired x-ray image beyond a degradation caused by the detected noise, wherein predicting optimal x-ray system settings comprises predicting with use of (i) an image simulator model G(x) 38 and (ii) a discriminator D 48. In one embodiment, the image simulator model G(x) 38 is trained to quantify a resulting loss or increase in image quality based on a given change in the x-ray system parameters in order to assist with selecting a magnitude of each parameter change. In another embodiment, the image simulator model G(x) 38 describes how a change in x-ray system settings would affect the current x-ray image, wherein the image simulator model G(x) receives as input (i) current x-ray system settings, (ii) the current x-ray image 34, and (iii) a set of new system settings 46 and produces as output 42 a new image that approximates an expected image given the change in x-ray system settings. Still further, in another embodiment, the image simulator model G(x) comprises a physics-based model or a neural network.

In accordance with another embodiment, the system and method further comprises optimizing the predicted optimal x-ray system settings output from the system settings model by (i) simulating, via the image simulator model G(x) 38, an expected x-ray image output 42 based upon the predicted optimal x-ray system settings 46, (ii) comparing, via the discriminator D 48, (ii) (a) the expected x-ray image output 42 with (ii) (b) an original x-ray image 34 corresponding to the current x-ray image, and (iii) changing the predicted optimal x-ray system settings into new x-ray system settings which reduce the radiation dose in a such a way that makes a resulting x-ray image substantially indistinguishable from the original x-ray image.

In another embodiment, information from the x-ray imaging system, as well as from the operating room (i.e., in which the x-ray imaging system is located), such as room sensor/camera data and system interaction information, are used independently or in combination with the first embodiment to predict user behavior. Certain types of user behavior may indicate that the current procedural phase or event does not require a high-quality image. For instance, if the physician exhibits behavior that indicates that he or she is simply looking for the right view of the gross anatomy without looking at precise details such as small vasculature or devices, this behavior may indicate to the algorithm that a system setting producing lower radiation output would be appropriate. These additional inputs may be passed into the noise classification network H(x) 32 in FIG. 4 alongside the image with modifications in network architecture, as appropriate.

In a further embodiment, the noise classification network H(x) 32 of FIG. 4 is a recurrent neural network which takes as input a series of images in a live fluoroscopy run as they (i.e., the individual images of the series) arrive, updating an internal hidden state with each new image in order to better predict changes in behavior, motion, or other temporal variables.

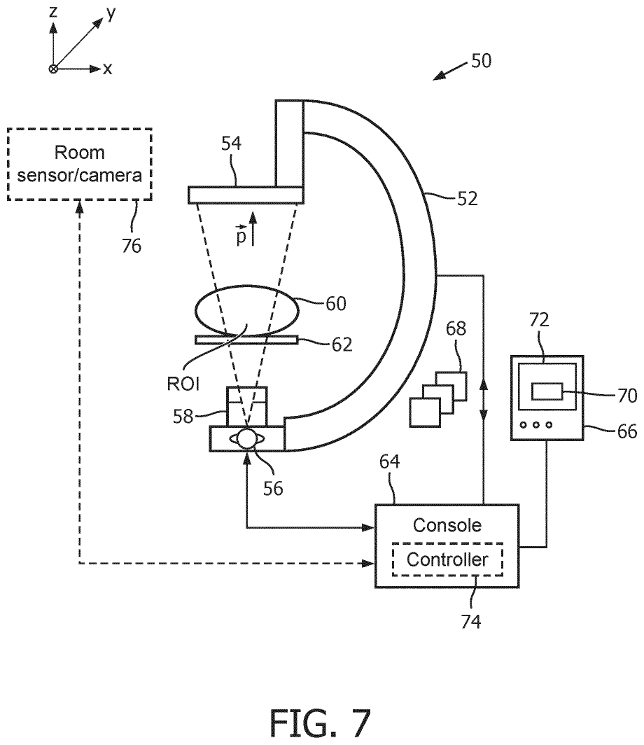

In still another embodiment, the noise classification network's outputs may be dependent on time since the obfuscating event occurred. For instance, when users are performing iso-centering with live fluoroscopy (i.e., moving the C-arm and/or table in order to center the region of interest), the rapid movement in the acquired fluoroscopy images should indicate that a lower image quality, and therefore lower dose, is acceptable. Suggested network changes, therefore, could be made in real time during iso-centering. However, once iso-centering is complete and new fluoroscopy images are being acquired sometime later, the influence of the obfuscating event on the x-ray imaging system settings for new image acquisition is lower. That is, the influence of obfuscating events on image x-ray imaging system settings may decay with time. In other words, an output of the noise detection neural network can be additionally dependent on a duration of time since any of the one or more noise-causing and/or user behavior events occurred. Modulations of the x-ray system parameters based on outputs of the noise detection neural network are implemented in real time during the detected one or more noise-causing and/or user behavior event. Once a respective event is complete or no longer occurring and new x-ray images are being acquired sometime later, an influence of the respective event on the x-ray system settings for new x-ray image acquisition is lower, having decayed with time, wherein a controller 74 (as will be discussed herein with respect to FIG. 7) reverts modulated x-ray system parameters back to parameters which existed prior to a respective modulation.

With reference now to FIG. 7, there is shown a block diagram view of an x-ray imaging system 50 configured for modulating radiation dosage according to an embodiment of the present disclosure. While FIG. 7 shows imager 50 of the C-arm type however it is understood that other imager constructions may also be put to use. Imager 50 includes a rigid C-arm 52 having affixed thereto at one of its ends a detector 54 and to the other an X-ray source (e.g., X-ray tube) 56 and a collimator 58 (hereinafter together referred to as the CX-assembly). X-ray source 56 operates to generate and emit a primary radiation X-ray beam p whose main direction is schematically indicted by vector p. Collimator 58 operates to collimate said X-ray beam in respect of a region of interest ROI within a patient 60 positioned upon an examination table 62.

The position of the C-arm 52 is adjustable so that the projection images can be acquired along different projection directions p. The C-arm 52 is rotatably mounted around the examination table 62. The C-arm 52 and with it the CX assembly is driven by a stepper motor or other suitable actuator (not shown).

Overall operation of imager 50 is controlled by operator from a computer console 64. Console 64 is coupled to screen 66. Operator can control via said console 64 any one image acquisition by releasing individual X-ray exposures for example by actuating a joy stick or pedal or other suitable input means (not shown) coupled to the console 64. During an intervention and imaging, the examination table 62 (and with it patient 60) is positioned between detector 54 and X-ray source 56 such that the region of interest ROI is irradiated by the radiation beam.

Broadly, during an image acquisition, the collimated X-ray beam emanates from X-ray source 56, passes through patient 60 at the region ROI, experiences attenuation by interaction with matter therein, and the so attenuated beam then strikes the surface of detector 54 at a plurality of detector cells. Each cell that is struck by an individual ray (of the primary beam) responds by issuing a corresponding electric signal. The collection of said signals is then translated by a data acquisition system (“DAS”—not shown) into a respective digital value representative of said attenuation. The density of the organic material making up the ROI determines the level of attenuation. High density material (such as bone) causes higher attenuation than less dense materials (such as the vessel tissue). The collection of the so registered digital values for each X-ray are then consolidated into an array of digital values forming an X-ray projection image for a given acquisition time and projection direction.

During a given image controlled interventional procedure, the imager 50 is operated to acquire a sequence of “live” fluoroscopic X-ray projection images 68 (“fluoros”) or angiograms (“angios”) during the given interventional procedure. In addition, during the interventional procedure, a user may initiate or specify various procedural steps via keystroke input or via a graphical user interface (GUI) widget 70 such as a drop-down menu or other graphical input arrangement or otherwise. For example, the GUI widget 70 may appear over laid a current acquired x-ray image 72 of the sequence of images 68

With reference still to FIG. 7, the system 50 further comprises a controller 74 located within the console 64. In one embodiment, controller 74 comprises one or more of a microprocessor, microcontroller, field programmable gate array (FPGA), integrated circuit, discrete analog or digital circuit components, hardware, software, firmware, or any combination thereof, for performing various functions as discussed herein, further according to the requirements of a given interventional x-ray imaging system implementation and/or application for modulating radiation dosage as discussed in the present disclosure.

Controller 74 can further comprise one or more of various modules configured for implementing one or more respective steps of the method for modulating radiation dosage as discussed herein, according to one or more embodiment of the present disclosure. For example, one module may include a room sensor/camera module which functions to capture inputs from a room sensor, camera, or both (collectively indicated via reference numeral 76 in FIG. 7). Such room sensor or camera inputs would be captured for use in one or more embodiment of the method for modulating radiation dosage as discussed herein.

It is understood that the one or more various modules may be computer program modules which are rendered in a non-transitory computer-readable medium. In one embodiment, a non-transitory computer-readable medium is encoded with computer program code that comprises a set of instructions, executable by a computer for enabling the computer to carry out the method to modulate radiation dosage based upon detection of at least one noise-causing event or user behavior event during performance of an x-ray-guided procedure with an x-ray imaging system, as discussed herein with respect to the various embodiments of the present disclosure.

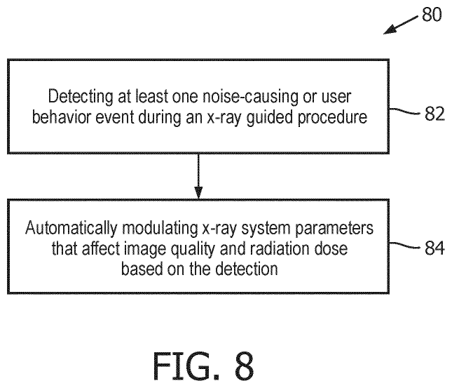

With reference now to FIG. 8, there is shown a flow diagram view of a method 80 for modulating radiation dosage according to an embodiment of the present disclosure. The method for modulating radiation dosage comprises: detecting (Step 82) at least one noise-causing event or user behavior event, during an x-ray guided procedure with an x-ray imaging system, that renders an image quality for x-ray imaging above a threshold image quality unnecessary; and automatically modulating (Step 84) x-ray imaging system parameters that affect image quality and radiation dose based on the detection of the at least one of the noise-causing event or user behavior event. In one embodiment, the method further includes at least one of (a) wherein the detecting comprises detecting via a noise detection model that is designed to detect the at least one noise-causing event or user behavior event, during the x-ray guided procedure, or (b) wherein automatically modulating the x-ray system parameters comprises modulating according to a system settings model trained to control x-ray system settings based on an output from the detecting step. In one example embodiment, the noise detection model and system setting model is combined into a single model.

Although only a few exemplary embodiments have been described in detail above, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. Combinations of the above embodiments, and other embodiments not specifically described herein, will be apparent to those of skill in the art upon reviewing the description. In the claims, means-plus-function clauses are intended to cover the structures described herein as performing the recited function and not only structural equivalents, but also equivalent structures. In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A system (50) for modulating radiation dosage, the system comprising:
a detector (32) configured to detect at least one noise-causing event or user behavior event, during an x-ray guided procedure with an x-ray imaging system (50), that renders an image quality for x-ray imaging above a threshold image quality unnecessary; and
a controller (74) configured to automatically modulate x-ray system parameters that affect image quality and radiation dose based on the detection of the at least one of the noise-causing event or user behavior event.

2. The system (50) according to claim 1, wherein the detector (32) comprises a noise detection model (30) that is designed to detect the at least one noise-causing event or user behavior event, during the x-ray guided procedure.

3. The system (50) according to claim 1, wherein the controller (74) is further configured to automatically modulate the x-ray system parameters according to a system settings model (44) designed to control x-ray system settings based on an output from the detector (32).

4. The system (50) according to claim 3, wherein the detector (32) comprises a noise detection model that is designed to detect the at least one noise-causing event or user behavior event, during the x-ray guided procedure.

5. The system (50) according to claim 4, wherein the system for modulating radiation dosage is trained by:
receiving X-ray image data containing noise from a noise-causing event,
inputting received X-ray image data into the noise detection model,
generating a predicted level of noise in the inputted image using the noise detection model,
inputting the predicted level of noise into the system settings model,
generating a predicted X-ray system parameters using the system settings model,
adjusting parameters of the noise detection model, or the system settings model, or both, based on a comparison between:
(i) the predicted level of noise with an expected level of noise, or
(ii) a simulated image generated from the predicted X-ray system parameters with an expected X-ray image, or
(iii) a combination thereof, and
repeating the inputting, the generating, and the adjusting, until a stopping criterion is met.

6. The system (50) according to claim 2, wherein the noise detection model (32) includes a noise classifier H(x) configured for detecting noise levels from different noise sources, wherein noise of the noise levels refers to any source of obfuscation in a current x-ray image of the x-ray guided procedure.

7. The system (50) according to claim 6, wherein the noise classifier H(x) is trained via producing a training data set in which known motion, obscuration, user behaviors, and other augmentations were introduced while using the x-ray imaging system.

8. The system (50) according to claim 6, wherein the controller (74) is further configured to automatically modulate the x-ray system parameters according to a system settings model (44), wherein the system settings model is designed to control x-ray system settings according to a set of predicted optimal x-ray system settings based on an output from the noise classifier H(x) of the noise detection model (32), wherein the image quality is reduced to a visually perceivable threshold level that is indistinguishable or nearly indistinguishable from the original image in the presence of the detected noise.

9. The system (50) according to claim 8, wherein the system settings model (44) comprises at least one selected from the group consisting of (i) a direct noise level to system setting mapping function that directly maps a set of detected noise levels to the set of predicted optimal x-ray system settings, (ii) a semi-supervised image based mapping of noise level to system settings that is trained without directly labelling ideal x-ray system settings for each input training image or behavior, and (iii) a combination thereof, to provide the set of predicted optimal x-ray system settings.

10. The system (50) according to claim 1, wherein the detector (32) is further configured to predict a user behavior, indicative of a current procedural phase or event of the x-ray-guided procedure being performed via the x-ray imaging system, that does not require an x-ray image having a quality greater than a threshold image quality, based upon information inputs from (i) the x-ray imaging system and (ii) an operating room in which the x-ray imaging system is located, the information inputs including at least one of (a) the current x-ray image, (b) x-ray imaging system user interaction information, and (c) operating room sensor/camera data.

11. The system (50) according to claim 2, wherein the noise detection model (32) takes as input a series of x-ray images in a live fluoroscopy run as individual images of the series are acquired, and considers the series of x-ray images in parallel with each newly acquired x-ray image in order to better predict changes in behavior, motion, or other temporal variables.

12. The system (50) according to claim 2, wherein an output of the noise detection model (32) is additionally dependent on a duration of time since any of the at least one noise-causing or user behavior event occurred, wherein modulations of the x-ray system parameters based on outputs of the noise detection model are implemented in real time during the detected at least one noise-causing or user behavior event and once a respective event is complete or no longer occurring and new x-ray images are being acquired sometime later, an influence of the respective event on the x-ray system settings for new x-ray image acquisition is lower, having decayed with time, wherein the controller reverts modulated x-ray system parameters back to parameters which existed prior to a respective modulation.

13. The system (50) according to claim 1, further including at least one of (a) wherein the at least one noise-causing or user behavior event comprises (i) an obfuscation or motion noise-causing event or (ii) a user behavior event requiring an image quality less than or equal to the threshold image quality, associated with respect to a current x-ray image of the x-ray guided procedure, or (b) wherein the x-ray system parameters comprise at least one selected from the group consisting of kV/kVp, mA, exposure time, frame rate, magnification, and any combination thereof.

14. The system (50) according to claim 13, wherein the obfuscation noise-causing event includes at least an object obscuring a field of view of an x-ray tube of the x-ray imaging system, wherein the motion noise-causing event includes at least a patient or an x-ray tube or x-ray detector component movement, and wherein the user behavior event includes at least a view-finding event.

15. An x-ray imaging system (50), comprising:
a detector (32) configured to detect at least one noise-causing event or user behavior event, during an x-ray guided procedure, that renders an image quality for x-ray imaging above a threshold image quality unnecessary; and
a controller (74) configured to automatically modulate x-ray system parameters that affect image quality and radiation dose based on the detection of the at least one of the noise-causing event or user behavior event.

16. The x-ray imaging system (50) according to claim 15, wherein the detector (32) comprises a noise detection model that is designed to detect the at least one noise-causing event or user behavior event, during the x-ray guided procedure, and
wherein the controller (74) is further configured to automatically modulate the x-ray system parameters according to a system settings model designed to control x-ray system settings based on an output from the detector.

17. The x-ray imaging system (50) according to claim 16, wherein the x-ray imaging system is trained by:
receiving X-ray image data containing noise from a noise-causing event,
inputting received X-ray image data into the noise detection model,
generating a predicted level of noise in the inputted image using the noise detection model,
inputting the predicted level of noise into the system settings model,
generating a predicted X-ray system parameters using the system settings model,
adjusting parameters of the noise detection model, or the system settings model, or both, based on a comparison between:
(i) the predicted level of noise with an expected level of noise, or
(ii) a simulated image generated from the predicted X-ray system parameters with an expected X-ray image, or
(iii) a combination thereof, and
repeating the inputting, the generating, and the adjusting, until a stopping criterion is met.

18. A method for modulating radiation dosage, the method comprising:
detecting at least one noise-causing event or user behavior event, during an x-ray guided procedure with an x-ray imaging system, that renders an image quality for x-ray imaging above a threshold image quality unnecessary; and
automatically modulating x-ray system parameters that affect image quality and radiation dose based on the detection of the at least one of the noise-causing event or user behavior event.

19. The method according to claim 18, further including at least one of (a) wherein the detecting comprises detecting via a noise detection model that is designed to detect the at least one noise-causing event or user behavior event, during the x-ray guided procedure, or (b) wherein automatically modulating the x-ray system parameters comprises modulating according to a system settings model trained to control x-ray system settings based on an output from the detecting step.

20. A non-transitory computer-readable medium encoded with computer program code that comprises a set of instructions, executable by a computer for enabling the computer to carry out the method according to claim 18, to modulate radiation dosage based upon detection of at least one noise-causing event or user behavior event during performance of an x-ray-guided procedure with an x-ray imaging system.
